(19) United States
(12) Patent Application Publication
(10) Pub. No.: US 2013/0190641 A1
(43) Pub. Date: Jul. 25, 2013

(54) MODULAR ACOUSTIC SPIROMETER

(71) Applicants: Lior Gonnen, Tel-Aviv (IL); David Groberman, Even Yehuda (IL); David Solomon, Zichron Ya'akov (IL); Rahav Cohen, Raanana (IL)

(72) Inventors: Lior Gonnen, Tel-Aviv (IL); David Groberman, Even Yehuda (IL); David Solomon, Zichron Ya'akov (IL); Rahav Cohen, Raanana (IL)

(73) Assignee: MEYTAR (DITAL) ENGINEERING LIMITED, Netanya (IL)

(21) Appl. No.: 13/825,146

(22) PCT Filed: Sep. 21, 2011
(86) PCT No.: PCT/IB11/54141

§ 371 Date: Mar. 20, 2013

Related U.S. Application Data
(60) Provisional application No. 61/385,166, filed on Sep. 22, 2010.

(51) Int. Cl. A61B 7/00

(52) U.S. Cl. CPC A61B 7/003
USPC 600/538

(57) ABSTRACT

A system for measuring spirometric flow rate using a microphone and a mobile computer device connected to or integrated with the microphone. The mobile computer device may include a processor and storage. The storage may be operatively connectible, e.g over a network, to a computer system used by a practitioner. The system has a transducer adapted for converting spirometric flow rate into an audible signal with an audio frequency characteristic of the spirometric flow rate. The microphone is external to the transducer and may be adapted to detect the audible signal having an audio frequency and to convert the audible signal to a corresponding electrical signal having the audio frequency. The audio frequency may be characteristic of a peak expiratory flow rate and the output result is characteristic of the peak expiratory flow rate.

### BACKGROUND

1. Technical Field

The present disclosure relates to spirometry.

2. Description of Related Art

Spirometry is a common pulmonary function test for measuring lung function. Specifically, spirometry is the measurement of the amount (volume) and/or speed (flow) of air that is inspired (inhaled) and/or expired (exhaled). Spirometry is an important technique in disease management for bronchial asthma and chronic obstructive pulmonary disease and other respiratory disorders.

The peak expiratory flow (PEF), an example of spirometric flow, is a person's maximum expiration flow rate, as measured with a peak flow meter, a small, hand-held device used to monitor a person's ability to breathe out air. The peak flow meter measures the degree of obstruction in the airways.

Spirometric monitoring may only be available at a clinic in which a doctor or nurse is located. The length between visits may be several months during which there is no monitoring of the patient's pulmonary function. The practitioner may be unaware of lifestyle factors: diet, exercise, monthly rhythms, daily circadian rhythms, introduction of new medications and withdrawal of medications and other factors which may result in a less than ideal treatment.

The introduction of telecommunications technologies in the healthcare environment has led to an increased accessibility to healthcare providers, more efficient tasks, processes and a higher overall quality improvement of healthcare services.

There is thus a need and it would be useful to have a modular spirometric flow rate measurement system and/or a peak expiratory flow measurement system which may be used by the patient to monitor and store and/or upload spirometric flow rate and/or peak flow measurement results to provide to the medical practitioner.

### BRIEF SUMMARY

According to an aspect of the present disclosure there is provided a system for measuring spirometric flow rate using a microphone and a mobile computer device connected to or integrated with the microphone. The mobile computer device may include a processor and storage. The storage may be operatively connectible, e.g over a network, to a computer system used by a practitioner. The system has a transducer adapted for converting spirometric flow rate into an audible signal with an audio frequency characteristic of the spirometric flow rate. The transducer may include a fluidic oscillator, a mechanical siren, a shredded vortex generator or a pressure orifice. The transducer may be mechanically attachable externally to the microphone to mitigate audio noise received by the microphone. The transducer may avoid physical and electrical connection and electromagnetic coupling to the microphone. The microphone is external to the transducer and may be adapted to detect the audible signal having an audio frequency and to convert the audible signal to a corresponding electrical signal having the audio frequency. The audio frequency may be characteristic of a peak expiratory flow rate and the output result is characteristic of the peak expiratory flow rate.

The system may include a computer readable medium which includes instructions executable by a processor. The instructions may be operable to enable the processor: to receive the electrical signal, to process the electrical signal, to derive the characteristic frequency and to output a result characteristic of the spirometric flow rate responsive to the audio frequency.

The instructions may be configured to schedule spirometric tests responsive to prior spirometric test results. The instructions may include for deriving the audio frequency a signal processing algorithm which may be a fast Fourier transform (FFT), an auto-correlation function, adaptive-additive algorithm, discrete Fourier transform, Bluestein's FFT algorithm, Bruun's FFT algorithm, Cooley-Tukey FFT algorithm, Prime-factor FFT algorithm, Rader's FFT algorithm or a fast folding algorithm.

According to an aspect of the present disclosure there is provided a method for measuring spirometric flow rate which uses a mobile computer device. The mobile computer device may include a processor, a memory and a microphone. The method transduces spirometric flow rate into an audible signal having an audio frequency characteristic of the spirometric flow rate. The audible signal may be converted to a corresponding electrical signal having the audio frequency. A transducer may be provided to perform the converting. The processor may be enabled to receive the electrical signal, to process the electrical signal to derive the audio frequency and to output the spirometric flow rate responsive to the audio frequency. The method may be characterized by the audible signal being received by using the microphone of the mobile computer device. The microphone may be external to the transducer thereby avoiding a cable and avoiding a wireless interface between the mobile computer device and the transducer. Scheduled spirometric tests may be enabled which are responsive to prior spirometric test results. Uploading of spirometric test results together with time stamp and user selectable parameters to a computer system in use by a practitioner may be also enabled. The processor may be enabled to record ambient noise. Upon the ambient noise being higher than a threshold, the processor may alert the user to postpone the use of the transducer, e.g. exhalation by the user or to use the transducer again after the ambient noise level has decreased below the threshold.

According to an aspect of the present disclosure there is provided a transducer adapted for converting spirometric flow into an audible signal. The transducer has a stator including a including a stator plate with a plurality of stator holes. A rotor is rotatably connected to the stator. The rotor may include multiple rotor blades configured to rotate the rotor responsive to the spirometric flow. The transducer avoids having a microphone. The audible signal is received externally to the transducer while avoiding a cable connection thereto. The audible signal includes an audio frequency responsive to the spirometric flow. The rotor may include a rotor plate with multiple rotor holes. The spirometric flow through the stator holes and the rotor holes substantially only when the stator holes and rotor holes are at least partially aligned. The audible signal has a characteristic audio frequency responsive to the spirometric flow produced by chopping spirometric flow between the stator plate and the rotor plate. The microphone is external to the transducer and may be adapted to detect the audible signal having an audio frequency and to convert the audible signal to a corresponding electrical signal having the audio frequency.

According to an aspect of the present disclosure there is provided a computer readable medium including instructions executable by a mobile computer device. The mobile computer device may include a processor and a microphone for use in a system for measuring a spirometric flow rate. The system may include a transducer adapted to transduce a spirometric flow rate into an audible signal with an audio frequency into an audible signal characteristic of the spirometric flow rate. The microphone receives the audible signal. The microphone is external to the transducer, thereby avoiding a cable between the mobile computer device and the transducer. The computer readable medium comprises instructions executable by the processor. The instructions are operable to enable the processor: to receive an electrical signal from the microphone responsive to the audio frequency, to process the electrical signal to derive the frequency and to output e, e.g. display, store, and/or transfer over the Internet, the spirometric flow rate responsive to the audio frequency.

### BRIEF DESCRIPTION OF THE DRAWINGS

The invention is herein described, in a non-limiting manner, by way of example only, with reference to the accompanying drawings, wherein:

FIG. 1a shows a system diagram for measuring spirometric flow rate device using an acoustic transducer and a mobile computer device, according to an exemplary feature of the present invention.

FIG. 1b shows a wide area network (WAN) bi-directionally connectible to a mobile computer device, a server and a client.

FIGS. 2a, 2b and 2c show feature details and usage of an example of acoustic transducer based on a mechanical siren.

FIG. 2d shows an exemplary feature of an acoustic transducer.

FIG. 2e shows a bottom view of a mobile computing device.

FIGS. 3a, 3b and 3c show a number of further examples for a rotor.

FIG. 3d which shows a cross sectional plan view along an axis of a stator and a vent which includes a stator plate and a rotor.

FIG. 4 shows a method which may be used to measure spirometric flow rate using an acoustic transducer and a mobile computer device, according to an exemplary feature.

The foregoing and/or other aspects will become apparent from the following detailed description when considered in conjunction with the accompanying drawing figures.

### DETAILED DESCRIPTION

Reference will now be made in detail to features of the present invention, examples of which are illustrated in the accompanying drawings, wherein like reference numerals refer to the like elements throughout. The features are described below to explain the present invention by referring to the figures.

The features of the present invention may comprise a general-purpose or special-purpose computer system including various computer hardware components, which are discussed in greater detail below. Features within the scope of the present invention also include computer-readable media for carrying or having computer-executable instructions, computer-readable instructions, or data structures stored thereon. Such computer-readable media may be any available media, which is accessible by a general-purpose or special-purpose computer system. By way of example, without limitation, such computer-readable media can comprise physical storage media such as RAM, ROM, EPROM, flash disk, CD-ROM or other optical disk storage, magnetic disk storage or other magnetic storage devices, or any other media which can be used to carry or store desired program code means in the form of computer-executable instructions, computer-readable instructions, or data structures and which may be accessed by a general-purpose or special-purpose computer system. Computer-readable media may include a computer program or computer application downloadable to the computer system over a network, such as a wide area network (WAN), e.g. Internet.

In this description and in the following claims, a “computer system” is defined as one or more software modules, one or more hardware modules, or combinations thereof, which work together to perform operations on electronic data. For example, the definition of computer system includes the hardware components of a personal computer, as well as software modules, such as the operating system of the personal computer. The physical layout of the modules is not important. A computer system may include one or more computers coupled via a computer network. Likewise, a computer system may include a single physical device (such as a phone or Personal Digital Assistant “PDA”) where internal modules (such as a memory and processor) work together to perform operations on electronic data. While any computer system may be mobile, the term “mobile computer system” or the term “mobile computer device” as used herein especially includes laptop computers, netbook computers, cellular telephones, smart phones, wireless telephones, personal digital assistants, portable computers with touch sensitive screens and the like.

The term “server” as used herein refers to a computer system including a processor, data storage and a network adapter generally configured to provide a service over the computer network. A computer system which receives a service provided by the server may be known as a “client” computer system.

In this description and in the following claims, a “network” is defined as any architecture where two or more computer systems may exchange data. The term “network” may include wide area network, Internet local area network, Intranet, wireless networks such as “Wi-fi”, virtual private networks, mobile access network using access point name (APN) and Internet. Exchanged data may be in the form of electrical signals that are meaningful to the two or more computer systems. When data is transferred or provided over a network or another communications connection (either hardwired, wireless, or a combination of hardwired or wireless) to a computer system or computer device, the connection is properly viewed as a computer-readable medium. Thus, any such connection is properly termed a computer-readable medium. Combinations of the above should also be included within the scope of computer-readable media. Computer-executable instructions comprise, for example, instructions and data which cause a general-purpose computer system or special-purpose computer system to perform a certain function or group of functions.

The articles “a”, “an” is used herein, such as “a processor”, “a server”, a “sample” have the meaning of “one or more” that is “one or more processors”, “one or more servers” and “one or more samples”.

Before explaining features of the invention in detail, it is to be understood that the invention is not limited in its application to the details of design and the arrangement of the components set forth in the following description or illustrated in the drawings. The invention is capable of other features or of being practiced or carried out in various ways. Also, it is to be understood that the phraseology and terminology employed herein is for the purpose of description and should not be regarded as limiting.

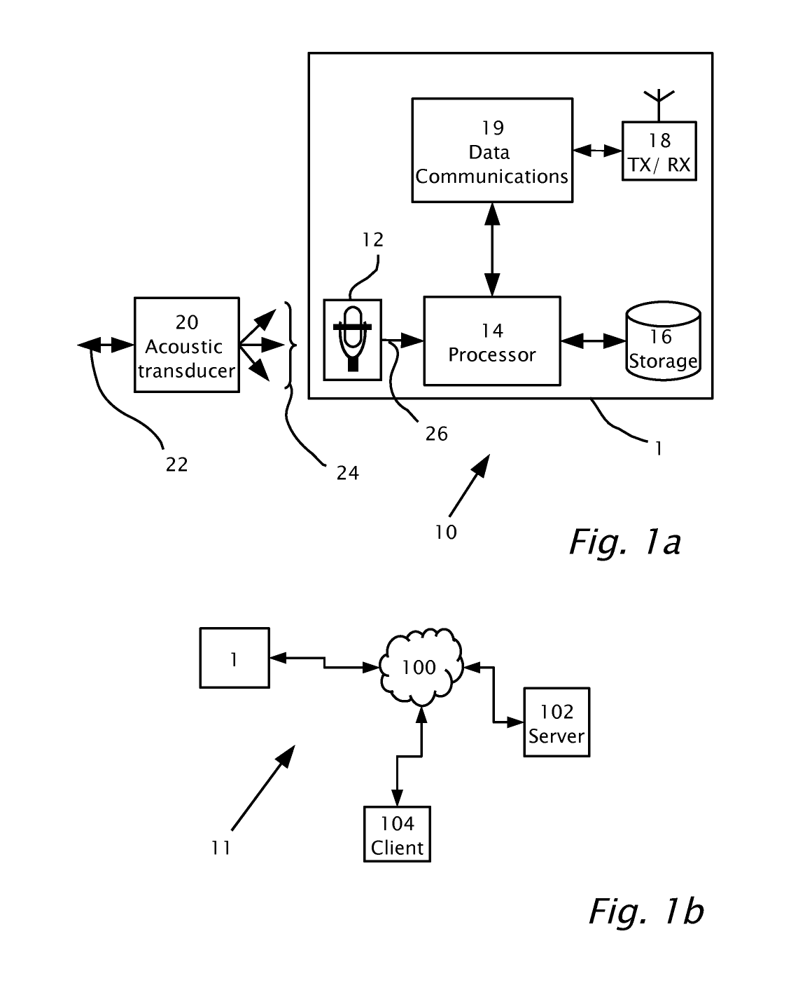

Referring now to the drawings, FIG. 1a shows a system diagram 10 for measuring spirometric flow rate device using an acoustic transducer 20 and a mobile computer device 1, according to an exemplary feature of the present invention. A user of mobile computer device 1 exhales and/or inhales through acoustic transducer 20 to undergo spirometric monitoring. Acoustic transducer 20 converts spirometric flow rate into an audible signal 24.

An example for acoustic transducer 20 may be based on known designs such as a fluidic oscillator shown in U.S. Pat. No. 4,244,230 in which a feedback loop creates vibrations at a frequency that is a function of pressure or flow rate. Audible sound 24 with a frequency range from 100 Hertz to 32 KiloHertz from transducer 20 may be optimal for microphone 12 at typical flow rates of 50 to 800 liters per minute.

Another example for acoustic transducer 20 may be in which air flow produces a shredded vortex in a frequency that is proportional to flow rate. The generated audible sound 24 from the shredded vortex may not be very strong when compared to other acoustic devices 20 and may also may have broader spectrum of audio frequencies.

Another example for acoustic transducer 20 may be based on an electronic acoustic device based on any kind of flow meter that has electronic output that modulates an acoustic frequency generator having a monotonous characteristics of flow rate (50-800 l/min) vs. frequency the range of 50 to 3000 Hz. Optionally the acoustic device may produce different frequency range for inspiration and expiration. The flow meter may include for instance an orifice plate placed in the path of airflow. An orifice plate is a device used for measuring the volumetric flow rate. It uses the same principle as a Venturi nozzle, namely Bernoulli's principle which states that there is a relationship between the pressure of the air and the velocity of the air. When the velocity increases, the pressure decreases and vice versa. From a sensed difference in air pressure before the orifice plate and after the orifice plate, the volumetric and mass flow rates may be obtained from Bernoulli's equation. For transducer 20 audible sound 24 generation may be performed by a microprocessor with an analog input of the sensed difference at the orifice which generates audible sound 24 corresponding to the sensed difference.

A possible standard to follow and apply with respect to spirometer design may be British standard BS EN ISO 23747-2009. British standard BS EN ISO 23747-2009 entitled “Anaesthetic and respiratory equipment. Peak expiratory flow meters for the assessment of pulmonary function in spontaneously breathing humans” deals with Anaesthetic equipment, Flow meters, Anaesthesiology, Medical breathing apparatus, Electrical medical equipment, Medical equipment, Respiratory system, Flow measurement, Measuring instruments, Safety devices and Performance.

Referring back to FIG. 1 a and system diagram 10, computer device 1 may include a processor 14 and connected thereto a display 17, a microphone 12, a data communications unit 19, storage 16, a cellular telephone transceiver (not shown) and/or data transceiver 18. Microphone 12 may be located internally within device 1, on the external surface of device 1, or externally connected to mobile computer device through an audio input. Microphone 12 is operable to detect audible signal 24 having an audio frequency responsive to the spirometric flow. Processor 14 is operable to receive an analogue or digitized electrical signal 26 responsive to the spirometric flow rate and based on the frequency of audible signal 24 outputs a spirometric flow rate and optionally stores the flow rate as data in storage 16.

Processor 14 may include sample and hold circuitry and an analogue-to- digital converters (ADC) for converting audio electrical signal 26 to a corresponding digital signal. Storage 16 may store signal processing algorithms used by processor 14 for a signal processing of audio electrical signal 26. Results of the signal processing function of audio electrical signal 26 may be stored in storage 16 displayed or transferred to data communications unit 19.

The use of transducer 20 which outputs an acoustic signal avoids the requirement of a cable between mobile device 1 and transducer 20. Moreover, the use of radio frequency RF transceivers or wireless interface such as Blutooth™ may also avoided, according to features of the present invention.

Reference is now also made to FIG. 1b which shows a wide area network (WAN) 100 bi-directionally connectible to mobile computer device 1, a server 102 and a client 104. Server 102 may provide the exchange of information between client 104 and mobile computer device 1. Client 104 may belong to a practitioner including medical personnel, e.g. pulmonologist, nurse.

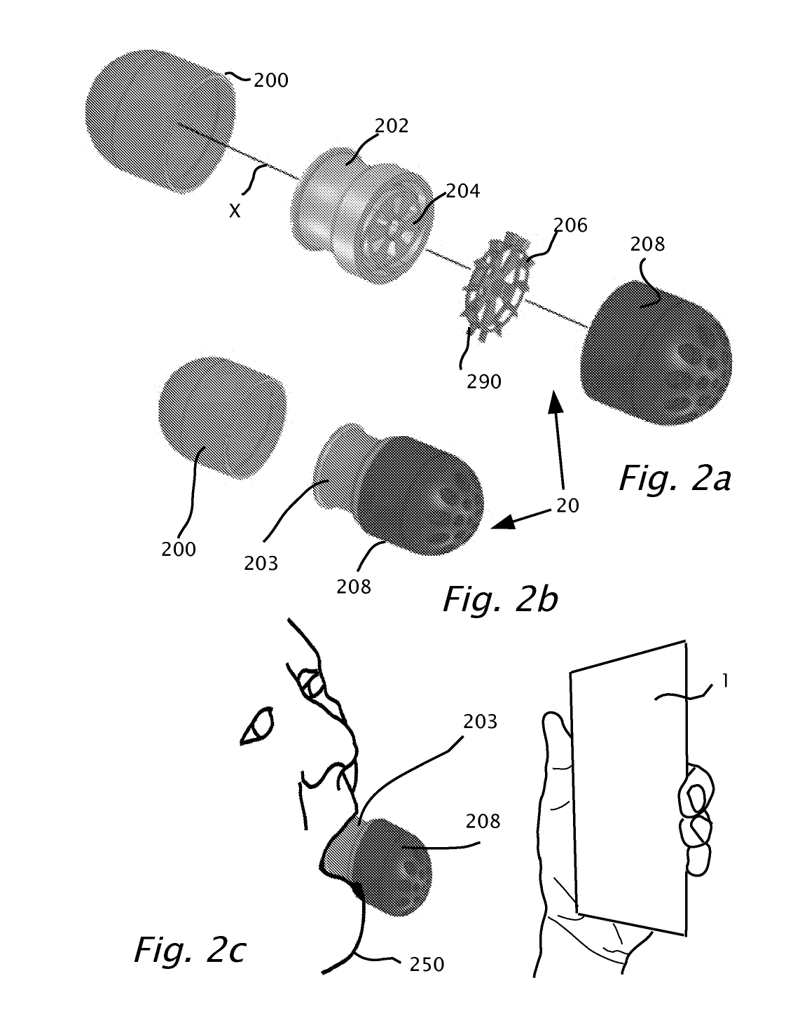

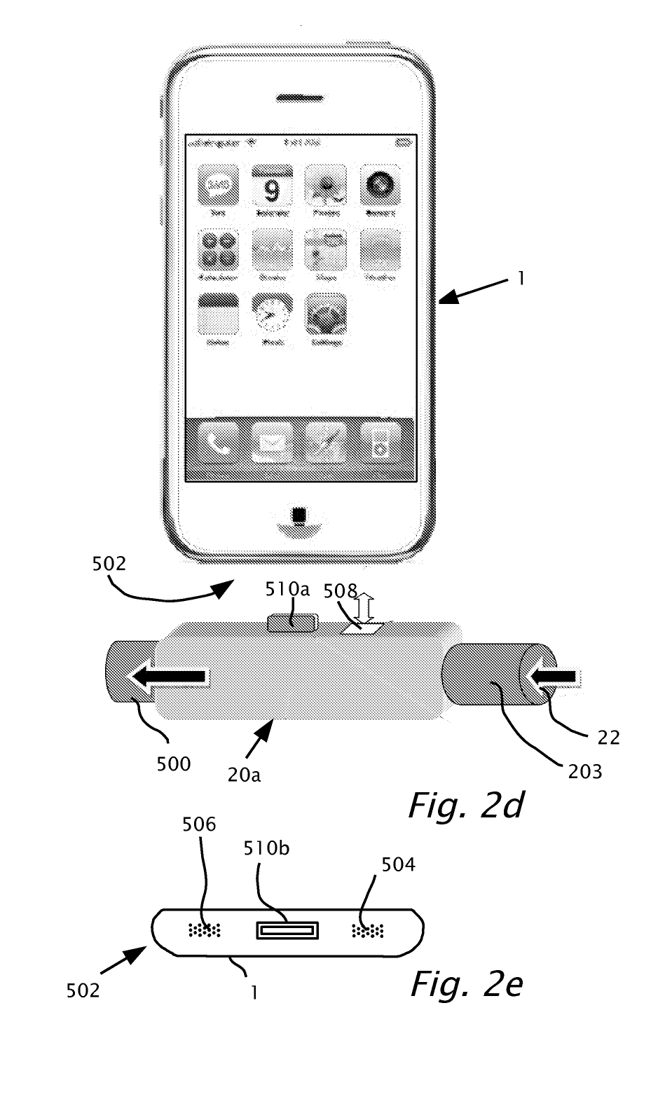

Reference is now made to FIGS. 2a, 2b and 2c which feature details and usage of an example of acoustic transducer 20 based on a mechanical siren. Transducer 20 includes a rotor 206, stator 202, vent 208 and protective cap 200.

A spindle may be located in the center of stator plate 204 and attached to the center of rotor 206, allows rotation of rotor 206 about axis X.

Rotation of rotor 206 occurs when the user exhales and/or inhales with the mouthpiece 203 sealed inside the mouth of the user during spirometric testing.

When spirometric air flow enters acoustic transducer 20, the air flows through the rotor blades which cause rotor 206 to rotate. Rotation of rotor 206 causes audible signal 24 to emanate from acoustic transducer 20 as spirometric air flows through mouthpiece aperture (not shown), through holes in stator 204, holes in rotor 206 and holes in vent 208. Stator plate 204 may have a number of slots or holes which may be similar in size and shape to the slots or holes of rotor plate of rotor 206. As rotor 206 rotates, audible signal 24 produced is by the chopping and consequent pressure variation of spirometric flow between the stator plate 204 and the rotor plate or rotor 206. When the holes in both plates are at least partially aligned air goes out through the holes and when the holes are not aligned the air flow is blocked. The transformation between open and blocked holes creates the chopping action. The chopped air flow and pressure the consequent variation create audible signal 24, the frequency of which frequency may depend on the rotational speed of the rotor. Higher rotation speed normal causes higher sound frequency. Consequently, audio electrical signal 26 has a characteristic audio frequency which is responsive to the spirometric flow rate produced by the chopping.

Audible signal 24 may be received by microphone 12 of mobile computer device 1. Mobile computer device 1 may be held in the vicinity of transducer 20 by the hand of the user as shown in FIG. 2c. Cap 200 may used to protect mouthpiece 203 when transducer is not being used. Cap 200 may also is not be used at all.

Reference is now made to FIG. 2e which shows a bottom view 502 of a mobile computing device 1 and to FIG. 2d which shows an exemplary feature of an acoustic transducer 20a. The acoustic transducer 20a has mouthpiece 203 which is placed in the mouth of a user. The user exhales and/or inhales to cause a spirometric air flow 22 through acoustic transducer 20a. Flow 22 continues through tube 500 on the opposite side to mouthpiece 202. Bottom 502 of mobile computing device 1 502 has two multiple holed through apertures 506 and 504 where built in speaker (not shown) and microphone 12 (not shown) of device 1 are located respectively. Bottom 502 may also have a female docking port 510b. Before exhaling and/or inhaling into mouthpiece 202. Transducer 20a may be mated with device 1 by inserting male docking port 510a of transducer 20a into female docking port 510b of device 1. Inserting male docking port 510a into female docking port 510b also aligns aperture 508 of transducer 20a with aperture 504, where built-in microphone 12 of device 1 is located. Inserting male docking port 510a into female docking port 510b followed by use of transducer 20a by the user may allow for non interference of unwanted noise sound external to transducer 20a and device 1. A fixed distance between audible signal 24 and microphone 12 may be achieved by repeated use of transducer 20a mated with device 1.

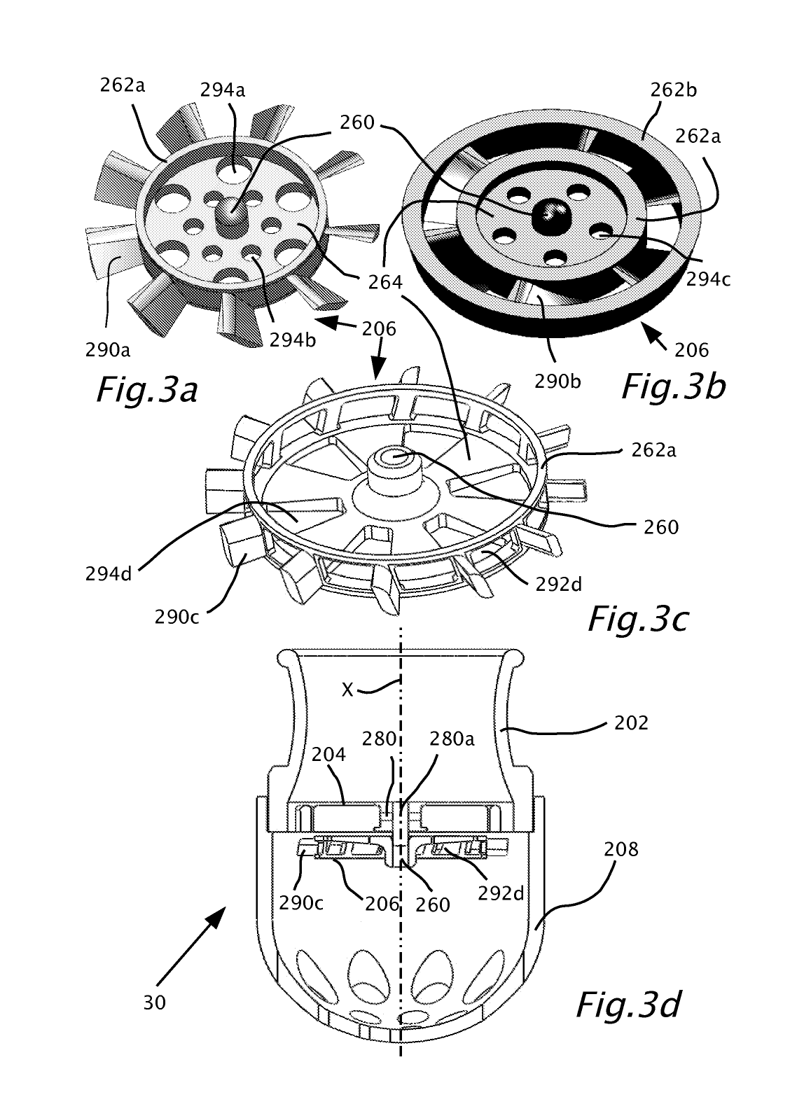

Reference is now made to FIGS. 3a, 3b and 3c which show a number of further examples for rotor 206. Different features for rotor 206 may produce different sound characteristics for audible signal 24 when rotor 206 is rotated. Common to rotors 206 in FIGS. 3a, 3b and 3c are blades 290, spindle housing 260, blade housing 262a and rotor plate 264. Blades 290 are configured to exert a rotational force on rotor 206 when spirometric air flows through transducer 20. Rotor plate 264 in FIG. 3a has larger circular holes 294a through the outer circumference of plate 264 and smaller circular holes 294b through the inner circumference of plate 264. Rotor plate 264 in FIG. 3b has circular holes 294c through the outer circumference of plate 264. Plate 264 in FIG. 3c has trapezoidal slots 294d which extend from the inner circumference to the outer circumference of plate 264. Blades 290a, 290b and 290c attach and protrude outwards from their respective blade housings 262a. The ends of blades 290b in FIG. 3b are additionally attached to an outer blade housing 262b. The blade housing 262a shown in FIG. 3c also has additional slots 292d between 290c blades.

Reference is now made to FIG. 3d which shows a cross sectional plan view 30 along axis X of stator 202 and vent 208 which includes stator plate 204 and rotor 206. In view 30, stator plate 204 is mounted at right angles to stator 202. Stator 202 includes a spindle 280a which runs along axis X. Spindle 280a is attached at one end to rotor housing 260 and the other end of spindle 280a located and attached in bearing 280. Bearing 280 allows the rotation of rotor 206 about axis X. Alternatively, bearing 280 may be located in rotor housing 260 and spindle 280a fixed at right angles to stator 206.

Rotor 206 and stator 202 may be designed so that the highest frequency of the sound at maximal spirometric flow is not noisy or disturbing to the user or to others. The lowest frequency should be high enough to be sensed by microphone 12.

For calculating the rotation speed of rotor 206, the air flow speed may be calculated in a first section (rotor blades section) which is proportional to a volumetric flow rate of the respiration. The volumetric flow rate being the percentage of the air which goes to the first section and the cross section area of the flow in the first section. The rotation speed may be proportional to the air flow speed in the rotor blades section and may depend on the angle and form of the rotor blades 290 of rotor 206.

The spirometer may measure a wide range of flow between 50 liters/min to 800 liters/minute. This wide range may be divided to give a device suitable for children and an adult device suitable for adults. The device suitable for children may have a low flow range L1 and for adults a high flow range L2.

The resistance pressure of the peak flow/spirometer has to be lower than 1.5 cm of water per 1 liter per second (for example for flow of 10 liters per second, the resistance pressure should be less than 15 cm of water).

The peak flow may measure transient peak flow during 0.1 second and therefore, the transducer preferably reaches a typical flow within 0.05 second.

Since the measurement of the spirometric flow may be performed by handset 1 with built in microphone 12, a transducer 20 may produce frequency and intensity that can be detected by the handset 1 and microphone 12 that is not attached to transducer 20. The following parameters may be suggested:

At low flow L1, the transducer 20 frequency may be more than 30 Hertz (Hz), (100 Hz may be optimal), as microphone 12 may be less sensitive to lower frequencies. While the resistance pressure of the peak flow/spirometer may be over 0.2 cm of water in order to produce a detectable audible sound 24 at low frequency.

At high flow rates L2, transducer 20 basic frequency (first harmonic) may be less than 18 Kilo Hertz (KHz), as microphone 12 may not be sensitive above 18 KHz. A design highest frequency of 3 KHz, may be set to decrease acceleration requirements for rotor 206 and reliability concerns.

Pressure can be estimated by the flow cross section (A) and flow rate using well known Bernoulli formula as a first approximation. Cross section (A) may be calculated from range L2 pressure limits or using 1.5 cm of water per 1 liter per minute.

The basic frequency equals to rotor 206 revolutions multiplied by the number of the openings in the rotor 206. The preferred number of openings may be 6 to 10. More openings may not be effective as a chopper. Fewer openings may increase the rotor frequency and may require higher acceleration.

Transducer 20 basic frequency (first harmonic) rotor frequency may be calculated by the angle of rotor blades 290 using known methods.

The number of blades 290 and profile of blades 290 may be optimized to supply the required torque that is required to provide acceleration requirements of reaching the maximal flow at 0.05 seconds.

The rotor 206 may be constructed with minimal moment of inertia , using low density and high strength polymers, for example polycarbonate.

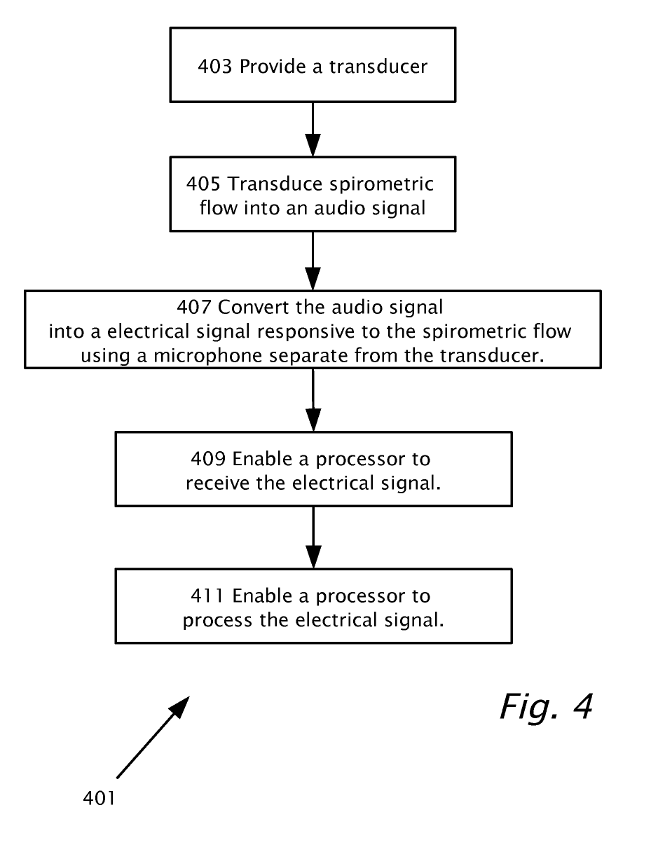

Reference is now made to FIG. 4 which shows a method 401 which may be used to measure spirometric flow rate using acoustic transducer 20 and mobile computer device 1, according to an exemplary feature. In step 403 transducer 20 is provided. The user inserts mouthpiece 203 into the mouth. The user exhales and/or inhales which causes spirometric air flow 22 through acoustic transducer 20. Airflow causes rotor 206 to rotate 20 which transduces the spirometric air flow to give audible signal 24 in step 405. Audible signal 24 is then converted into an electrical signal 26 by microphone 12 in step 407. Electrical signal 26 may be responsive to the spirometric air flow 22 through acoustic transducer 20. Processor 14 which may include sample and hold circuitry, analogue to digital converters (ADC), digital to analogue converters (DAC) and some working 25 memory which enables processor 14 to receive electrical signal 26 in step 409. After electrical signal 26 is received, processor 14 is also enabled to process electrical signal 26 in step 411. Processing of signal 26 in step 411 may be implemented by signal processing algorithms known in the art of signal processing. Results of signal processing step 411 may be displayed and/or stored (step 413) in storage 16 and/or sent (step 415) to data 30 communication unit 19 for transfer over wide area network 100, e.g. Internet.

Initialization of system 10 may include entering data such as age sex, target respiration values alert thresholds and medication schedules. Initialization may be performed by the user himself or by a practitioner located at a distant client 104 or server 102 (FIG. 1b). System 10 may usually be in a stand-by mode until the user chooses the spirometer measurement option or the stand-by mode may be entered by instructions via communication channels.

In a measurement mode operation, system 10 may provide notification for a start of a measurement. During the measurement, system 10 may record the characteristic frequency of acoustic device 20. The sound recording, sound analysis and frequency detection of audible sound 24 may be performed in real time on small time frames of 0.1 seconds in order to detect sound 24 and to identify sound 24. The signal analysis may be performed by a Fast Fourier Transform (FFT) to detect the maximum power at a particular frequency. The FFT may be performed in a defined frequency range eg. 100-2000 Hz for acoustic transducer 20 held at a distance away from mobile device 1 by a user. Since environmental noises may interfere with the measurement, a noise rejection procedure may be suggested to avoid artifacts. The system may use noise rejection criteria of having a characteristic transducer frequency (for example local maxima of the frequency in the range of 100-3000 hz) 3 to 4 times the surrounding environmental root mean squared (RMS) audio noise. In case that the particular (characteristic) frequency is not detected within 5-10 seconds, an error message may be presented and the user can start over the recording or close the application and system 10 goes into the stand-by mode.

The flow rate calculation may be performed in real time by software installed in mobile computer device 1 or after the recording session. The recording session ends after 5-10 seconds after the particular frequency detection or when the characteristic frequency is not detected. After the recording session is over, the flow rate may be calculated by using calibration data (from a function or a look-up table) of frequency versus flow rate. Additional respiratory parameters calculated from the flow rate curve as a function of time may be saved with a time stamp and optionally transferred to web-service management center, e.g. server 102.

An analysis of the respiratory parameters may include comparing the respiratory parameters to predefined values and in case of deviation from the normal values, an alert may be presented to the user with recommendations. The analysis may be performed in software of device 1 or in a facility that is supervised by a clinician. Device software and web servers software may work as one system, which may enable cost-effective supervision by the clinician and access of data by authorized users for disease management.

A scheduling of measurement may be performed by fixed schedule or by an adaptive regime. Fixed scheduling may be performed by programming time intervals between scheduled measurements or a time to measure as indicated to the user. Adaptive scheduling may be based on the trending information and scheduled intervals. While the measured respiratory values are in a normal range, a measurement may be performed at pre-scheduled intervals. When the measured spirometric measurement results are not normal, the measurement intervals may be decreased according to a predefined logic.

The application installable on device 1 may enable the entering of user selectable parameters and/or treatment parameters that can be correlated to the respiratory measurement results and displayed on the same chart, or uploaded to the practitioner for example. User selectable parameters may include: lifestyle factors: diet, exercise, monthly rhythms, daily circadian rhythms, introduction of new medications and withdrawal of medications A user selectable parameter that may be correlated over a time period of spirometric measurements of respiratory parameters for asthma for example, may be movement, e.g. aerobic exercise, of a user using over the time period using a mobile computer device 1 which includes a built-in acceleration sensor. System 10 can therefore be configured to present and display combined charts of breathing parameters with other measurable or manually entered parameters.

Although selected features of the present invention have been shown and described, it is to be understood the present invention is not limited to the described features. Instead, it is to be appreciated that changes may be made to these features without departing from the principles and spirit of the invention, the scope of which is defined by the claims and the equivalents thereof.

We claim:

1. A system for measuring spirometric flow rate using a microphone and a mobile computer device connected to or integrated with the microphone, wherein the mobile computer device includes a processor, the system comprising:
a transducer adapted for converting spirometric flow rate into an audible signal with an audio frequency characteristic of the spirometric flow rate wherein the microphone is external to said transducer and is adapted to detect said audible signal having said audio frequency and to convert said audible signal to a corresponding electrical signal having the audio frequency;
a computer readable medium including instructions executable by a processor, wherein said instructions are operable to enable the processor: to receive said electrical signal, to process the electrical signal to derive said audio frequency and to output as a result the spirometric flow rate responsive to said audio frequency.

2. The transducer of claim 1, wherein said transducer includes a component selected from the group consisting of: a fluidic oscillator, a mechanical siren, a shredded vortex generator and a pressure orifice.

3. The transducer of claim 1, wherein said transducer is mechanically attachable externally to said microphone, thereby mitigating audio noise received by said microphone.

4. The transducer of claim 1, wherein said transducer avoids physical and electrical connection and electromagnetic coupling to the microphone.

5. The transducer of claim 1, wherein said audio frequency is characteristic of a peak expiratory flow rate and the output result is characteristic of the peak expiratory flow rate.

6. The system of claim 1, wherein the mobile computer device includes storage, wherein the storage is operatively connectible to a computer system used by a practitioner.

7. The system of claim 1, wherein said instructions are configured to schedule spirometric tests responsive to prior spirometric test results.

8. The system of claim 1, wherein said instructions include for deriving said audio frequency a signal processing algorithm selected from the group consisting of: a fast Fourier transform (FFT), an auto-correlation function, adaptive-additive algorithm, discrete Fourier transform, Bluestein's FFT algorithm, Bruun's FFT algorithm, Cooley-Tukey FFT algorithm, Prime-factor FFT algorithm, Rader's FFT algorithm and a fast folding algorithm.

9. A method for measuring spirometric flow rate using a mobile computer device including a processor, a memory and a microphone, the method comprising:
transducing spirometric flow into an audible signal having an audio frequency characteristic of the spirometric flow rate;
converting said audible signal to a corresponding electrical signal having the audio frequency;
enabling the processor to receive said electrical signal, to process the electrical signal to derive said audio frequency and to output the spirometric flow rate responsive to said audio frequency;
wherein the method is characterized by:
receiving said audible signal using the microphone of the mobile computer device.

10. The method of claim 9, further comprising:
providing a transducer to perform said converting, wherein the microphone is external to said transducer thereby avoiding a cable and avoiding a wireless interface between the mobile computer device and the transducer.

11. The method of claim 9, further comprising:
enabling scheduling of spirometric tests responsive to prior stored spirometric test results.

12. The method of claim 9 further comprising:
enabling uploading of spirometric test results together with time stamp and user selectable parameters to a computer system in use by a practitioner.

13. The method of claim 9 further comprising:
enabling the processor to record ambient noise;
upon said ambient noise being higher than a threshold, enabling the processor to alert the user to postpone said transducing or to perform said transducing again.

14. A transducer adapted for converting spirometric flow rate into an audible signal, the transducer comprising:
a stator including a including a stator plate with a plurality of stator holes;
a rotor, rotatably connected to said stator, the rotor including a plurality of rotor blades configured to rotate said rotor responsive to the spirometric flow, characterised by:
avoiding having a microphone, whereby the audible signal is received externally to the transducer while avoiding a cable connection thereto, wherein the audible signal includes an audio frequency characteristic of the spirometric flow rate.

15. The transducer of claim 14, wherein the rotor includes a rotor plate with a plurality of rotor holes; wherein the spirometric flow flows through said stator holes and said rotor holes substantially only when said stator holes and rotor holes are at least partially aligned.

16. The transducer of claim 15, wherein said audible signal has a characteristic audio frequency responsive to the spirometric flow produced by chopping spirometric flow between said stator plate and said rotor plate.

17. The transducer of claim 14, wherein the microphone external to said transducer is adapted to detect said audible signal having an audio frequency and to convert said audible signal to a corresponding electrical signal having the audio frequency.

18. A computer readable medium including instructions executable by a mobile computer device including a processor and a microphone for use in a system for measuring a spirometric flow rate, the system including a transducer adapted to transduce a spirometric flow rate into an audible signal with an audio frequency characteristic of the spirometric flow rate, wherein the microphone receives the audible signal, wherein the microphone is external to said transducer thereby avoiding a cable between the mobile computer device and the transducer, the computer readable medium comprising: instructions executable by the processor, wherein said instructions are operable to enable the processor: to receive an electrical signal from the microphone responsive to said audio frequency, to process the electrical signal to derive said frequency and to output the spirometric flow rate responsive to said audio frequency.
